(19) United States
(12) Patent Application Publication
(10) Pub. No.: US 2016/0129252 A1
(43) Pub. Date: May 12, 2016

(54) SYSTEMS AND METHODS FOR NONINVASIVE ELECTRICAL BRAIN STIMULATION WITH POWER TUNES

(71) Applicant: Brainique AG, Stans (CH)

(72) Inventors: Raymond Watt, San Diego, CA (US); Jaen Frederik Labuschagne, Cape Town (ZA); John Sandhu, Boulder, CO (US)

(73) Assignee: Brainique AG, Stans (CH)

(21) Appl. No.: 14/896,479

(22) PCT Filed: Jun. 9, 2014
(86) PCT No.: PCT/EP2014/001559

§ 371 Date: Dec. 7, 2015

Related U.S. Application Data
(60) Provisional application No. 61/832,722, filed on Jun. 7, 2013.

(51) Int. Cl. A61N 1/36; A61N 1/04

(52) U.S. Cl. CPC A61N 1/36025; A61N 1/0456

(57) ABSTRACT

A method for providing noninvasive electrical brain stimulation “NIEBS” is provided, the method comprising: receiving a signal at a power converter wherein the signal is an audio signal; converting power from said audio signal to a form suitable for NIEBS; generating a NIEBS signal using said power converted from said audio signal; and applying NIEBS based on said able NIEBS signal to a user via electrodes.

### TECHNICAL FIELD

This application relates to systems and methods for noninvasive electrical brain stimulation with power tunes.

### BACKGROUND

Noninvasive Electrical Brain Stimulation (herein referred to as NIEBS) applies gentle micro-current pulses to the brain using electrodes. It is widely accepted that NIEBS stimulates the brain to manufacture neurotransmitters. Noninvasive electrical brain stimulation has also been proposed for treatment of various medical conditions.

The signals operate to normalize the electrical output of the brain. NIEBS has thus been used/tested to treat substance dependence, depression and anxiety. It has been noted in at least some instances that NIEBS has equal or greater efficacy for the treatment of depression when compared to antidepressant medications, with fewer side effects.

The mechanism by which NIEBS produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

### SUMMARY

According to a first aspect, there is provided a method for providing noninvasive electrical brain stimulation “NIEBS”, comprising: receiving a signal at a power converter wherein the signal is an audio signal; converting power from said audio signal to a form suitable for NIEBS; generating a NIEBS signal using said power converted from said audio signal; and applying NIEBS based on said NIEBS signal to a user via electrodes.

By providing the capability to convert power from an audio signal into a format suitable for NIEBS, it is possible to provide power for a NIEBS signal using standard audio equipment without the need for a dedicated power connection. Accordingly, the application of NIEBS can be simplified and/or made more efficient.

According to a second aspect, there is provided a device for applying noninvasive electrical brain stimulation “NIEBS”: a power converter for receiving an audio signal output from a device and converting a portion of the power from the audio signal output to a form suitable for NIEBS; a NIEBS generator for generating a NIEBS signal; and at least two electrodes for attaching to the skin of a user for applying NIEBS to said user based on said NIEBS signal using only power derived from said audio signal output.

By providing an apparatus that is capable of converting power from an audio signal to a form suitable for NIEBS, it is possible to receive treatment for NIEBS using an audio source as a power source without the need for a separate dedicated power source. Accordingly, the application of NIEBS can be simplified and/or made more efficient.

According to a third aspect, there is provided a method for providing noninvasive electrical brain stimulation “NIEBS”, comprising: receiving an audio signal wherein the audio signal comprises an audible portion and an inaudible portion; sending said audible portion is sent to at least one speaker to be played and said inaudible portion to a power converter; converting power from said inaudible portion to a power form suitable for NIEBS; generating a NIEBS signal based on said converted power; and applying NIEBS to a user via said NIEBS signal wherein said NIEBS is powered only by power derived from said audio signal.

By receiving an audio signal that includes both an audible portion and an inaudible portion, it is possible to combine audio content, such as music or speech, with a signal which cannot be heard by the listener. Accordingly, it is possible to integrate an inaudible signal for powering a NIEBS treatment into an audio signal without interfering with the audio transmitted to the user and without the need for a separate power cable. As such, the powering of a NIEBS signal can be provided through standard audio equipment. Accordingly, the application of NIEBS can be simplified and/or made more efficient.

According to a fourth aspect, there is provided an apparatus for providing noninvasive electrical brain stimulation “NIEBS” using a received audio signal comprising an audible portion and an inaudible portion, comprising: at least one speaker for playing said audible portion of said received audio signal; a power converter for converting power from said inaudible portion of said received audio signal to a power form suitable for NIEBS; a NIEBS generator for generating a NIEBS signal based on said converted power; electrodes for applying NIEBS to a user via said NIEBS signal wherein said NIEBS signal is powered only by power derived from said audio signal.

By receiving an audio signal that includes both an audible portion and an inaudible portion, it is possible to combine audio content with another signal which cannot be heard by the listener. Accordingly, it is possible to integrate an inaudible signal that is capable of providing electrical power for a NIEBS signal that can be used to treat a user. Specifically, an audio signal can be provided to the user through a loudspeaker without interference and power can be provided to the NIEBS generator without the need for an additional or separate connection for power. As such, the provision of power for generating a NIEBS signal can be provided through standard audio equipment, for example a standard microphone connection. Accordingly, the application of NIEBS can be simplified and/or made more efficient.

According to a fifth aspect, there is provided a method for creating a multiplexed audio program: receiving a request for a multiplexed audio program based on a modified audio track; obtaining an unmodified audio track at a mixer; mixing said unmodified audio track with a power tune to produce a multiplexed audio program.

By providing the capability to produce a multiplexed audio program, it is possible to include a power tune which can be used at an audio receiver to separate the power signal from an audio track so as to power a NIEBS generator whilst also providing the audio track without the need for a separate or additional cable for power. Accordingly, the application of NIEBS can be simplified and/or made more efficient.

According to a sixth aspect, there is provided a method for providing NIEBS, comprising: receiving a multiplexed audio program at a splitter from a device wherein the multiplexed audio program comprises an audible portion and an inaudible portion; splitting said multiplexed audio program at said splitter such that said audible portion is sent to speakers to be played and said inaudible portion is sent to a power converter; converting power from said inaudible portion to a power form suitable for NIEBS; generating a NIEBS signal based on said inaudible portion; and applying NIEBS to a user via said NIEBS signal wherein said NIEBS is powered only by power derived from said multiplexed audio program.

By providing the capability to receive and split a multiplexed audio program, it is possible to separate audible and inaudible portions of the audio program and thus utilize the audible portion to provide an audio signal to the user whilst also utilizing the inaudible portion of the audio program to provide electrical power to power a NIEBS signal for providing NIEBS to a user without the need for a separate cable and by using a standard audio source. Accordingly, the application of NIEBS can be simplified and/or made more efficient.

According to a seventh aspect, there is provided an apparatus for providing NIEBS, comprising: a splitter for receiving a multiplexed audio program at a splitter from a device wherein the multiplexed audio program comprises an audible portion and an inaudible portion; at least one speaker for playing said audible portion; a power converter for converting power from said inaudible portion to a power form suitable for NIEBS; a NIEBS generator for generating a NIEBS signal based on said inaudible portion; electrodes for applying NIEBS to a user via said NIEBS signal wherein said NIEBS is powered only by power derived from said multiplexed audio program.

By providing the capability to receive and split a multiplexed audio program, it is possible to separate audible and inaudible portions of the audio program and thus utilize the audible portion to provide an audio signal to the user whilst also utilizing the inaudible portion of the audio program to provide electrical power to power a NIEBS signal for providing NIEBS to a user without the need for a separate power source and by using a standard audio source. Accordingly, the application of NIEBS can be simplified and/or made more efficient.

According to an eighth aspect, there is provided a method for creating a multiplexed audio program: receiving a request from a user for a multiplexed audio program based on an modified audio track; obtaining said unmodified audio track at a mixer; mixing said unmodified audio track with a power tune at said mixer; and recording said mixing to create a multiplexed audio program.

By creating a multiplexed audio program that includes a power tune and an audio track it is possible to embed a power signal within an audio track and allow a remote device to utilize the power tune to power aspects of the remote device without the need for a separate power source or an additional power cable. Accordingly, the application of NIEBS can be simplified and/or made more efficient.

According to a ninth aspect, there is provided a method for downloading a NIEBS signal formulation in computer-readable format, comprising: providing a NIEBS signal formulation stored in a library available to a server via a processor; providing an internet connection from a processor; providing a NIEBS generator for executing a computer-readable code for creating a NIEBS signal; providing a data link between the NIEBS generator and the processor; providing a set of instructions for said processor to implement a data download and data transfer from internet to NIEBS generator.

By downloading a NIEBS signal formulation in a computer-readable format, it is possible to provide a NIEBS signal based upon a stored computer-readable format of NIEBS signal in an efficient manner. Accordingly, the application of NIEBS can be simplified and/or made more efficient.

According to a tenth aspect, there is provided a method for providing NIEBS, comprising: receiving an signal at a power converter wherein the signal is from a device and the signal is an audio source; converting power from said signal to a form suitable for NIEBS; generating a NIEBS signal using said power converted from said audio source; and applying NIEBS based on said NIEBS signal to a user via electrodes.

According to an eleventh aspect, there is provided a device for applying NIEBS: a power converter for receiving an audio signal output from a device and converting a portion of the power from the audio signal output to a form suitable for NIEBS; a NIEBS generator for generating a NIEBS signal; and at least two electrodes for attaching to the skin one either side of a user for applying NIEBS to said user based on said NIEBS signal using only power derived from said audio signal output.

According to a twelfth aspect, there is provided a method for providing NIEBS, comprising: receiving a two channel audio source at a splitter from a wired output of a device; splitting each of two channels associated with said two channel audio source into a first portion and second portion; passing said first portion to two speakers for playing audio of said two channel audio source; converting said second portion at a power converter to a form suitable for NIEBS; generating a NIEBS signal based on said second portion; and applying NIEBS to a user based on said NIEBS signal via electrodes associated with said two speakers wherein said electrodes are powered only by power derived from said wired output of said device.

According to a thirteenth aspect, there is provided an apparatus: a splitter for receiving a two channel audio output from a device via a wired output and for splitting each of said two channels and passing a first portion of each of said two channels to speakers for playing audio; an audio power converter for converting a second portion of each of said two channels to a form suitable for NIEBS; a NIEBS generator for generating a NIEBS signal based on said second portion; electrodes associated with said speakers for applying NIEBS to a user wherein said NIEBS is powered only by the power derived from said wired output of said device.

According to a fourteenth aspect, there is provided a method for providing NIEBS, comprising: providing a NIEBS generator having a pair of output ports; providing a first earpiece having a plurality of electrodes for delivering NIEBS signals from a first output port on said NIEBS generator to a user's skin in the region of his ear; providing an audio source for audible program material having two channels available with wired outputs; providing an audio power converter connected to a first channel of the audio source providing an audio output signal; and wherein the audio power converter converts alternating current audio signals into direct current to supply dc power to the NIEBS generator, and wherein the second audio output channel from said audio source is connected to a speaker in a second earpiece with a built-in speaker.

According to a fifteenth aspect, there is provided an apparatus for providing NIEBS, comprising: an audio source for audible program material having two channels available with wired outputs; an audio power converter connected to a first channel of the audio source providing an audio output signal; wherein the audio power converter converts alternating current audio signals from said first channel into direct current to supply dc power to a NIEBS generator, and wherein the second audio output channel from said audio source is connected to a speaker in a second earpiece with a built-in speaker; a NIEBS generator for receiving said dc power and for generating a NIEBS signal based on said first channel and having a pair of output ports; and a first earpiece having a plurality of electrodes for delivering NIEBS signals from a first output port on said NIEBS generator to a user's skin in the region of his ear.

### BRIEF DESCRIPTION OF THE DRAWINGS

FIG. 1A illustrates a block diagram of an example noninvasive electrical brain stimulation system in accordance with embodiments of the present technology.

FIG. 1B illustrates a block diagram of an example noninvasive electrical brain stimulation system in accordance with embodiments of the present technology.

FIG. 1C illustrates a block diagram of power converter and filter in accordance with embodiments of the present technology.

FIG. 1D illustrates a block diagram of an example environment for a noninvasive electrical brain stimulation system in accordance with embodiments of the present technology.

FIG. 1E illustrates a block diagram of an example environment for a noninvasive electrical brain stimulation system in accordance with embodiments of the present technology.

FIG. 1F illustrates a block diagram of an example environment for a noninvasive electrical brain stimulation system in accordance with embodiments of the present technology.

FIG. 2A illustrates a block diagram of an example environment for a noninvasive electrical brain stimulation system in accordance with embodiments of the present technology.

FIG. 2B illustrates a block diagram of an example environment for a noninvasive electrical brain stimulation system in accordance with embodiments of the present technology.

FIG. 2C illustrates a block diagram of an example environment for a noninvasive electrical brain stimulation system in accordance with embodiments of the present technology.

FIG. 3A illustrates a block diagram of an example environment for a noninvasive electrical brain stimulation system in accordance with embodiments of the present technology.

FIG. 3B illustrates a block diagram of a programmable noninvasive electrical brain stimulation system in accordance with embodiments of the present technology.

FIG. 3C illustrates a block diagram of an example environment for updating a noninvasive electrical brain stimulation system in accordance with embodiments of the present technology.

FIG. 3D illustrates a block diagram of an example environment for updating a noninvasive electrical brain stimulation system in accordance with embodiments of the present technology.

FIG. 3E illustrates a block diagram of an example integrated PDA/NIEBS system with updating in accordance with embodiments of the present technology.

FIGS. 4A-C, FIGS. 5A-C, and FIG. 6 illustrate block diagrams of example speakers and electrodes for use in a noninvasive electrical brain stimulation system in accordance with embodiments of the present technology.

FIG. 7 illustrate block diagrams of example pulse trains for use in a noninvasive electrical brain stimulation system in accordance with embodiments of the present technology.

FIG. 8 illustrates block diagrams of example waveforms for use in a noninvasive electrical brain stimulation system in accordance with embodiments of the present technology.

FIG. 9 illustrates a block diagram of an example noninvasive electrical brain stimulation system in accordance with embodiments of the present technology.

The drawings referred to in this description of embodiments should be understood as not being drawn to scale except if specifically noted.

### DETAILED DESCRIPTION

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

### Overview of Systems and Methods for Noninvasive Electrical Brain Stimulation with Power Tunes

Embodiments of the present technology are for systems and methods for noninvasive electrical brain stimulation with power tunes. The description and claims herein specifically describe noninvasive electrical brain stimulation (NIEBS). However, the present technology applies generally to electrotherapy and electro medicine in its many forms. Therefore, the descriptions and claims related to NIEBS may be extended to include electrotherapy in general. Types of electrotherapy may be for, but are not limited to, electro neurostimulation, electro neuromodulation, neuromodulation, brain stimulation, electro medicine, bone growth, muscle stimulation, pain management, etc.

For example, neurostimulation involves modulation of the nervous system and electrically activate neurons in the body. The activation of neural elements in a part of the nervous system can be effectively facilitated by stimulation. Micro-electrodes are utilized to interface with excitable tissue in order to either restore recording experiences to the implant recipient or control an effector organ. Additionally, neuromodulation is the physiological process by which a given neuron uses several different neurotransmitters to regulate diverse populations of central nervous system neurons. This is in contrast to classical synaptic transmission, in which one presynaptic neuron directly influences a single postsynaptic partner. Neuromodulators secreted by a small group of neurons diffuse through large areas of the nervous system, affecting multiple neurons. Examples of neuromodulators include dopamine, serotonin, acetylcholine, histamine and others.

Noninvasive electrical brain stimulation (NIEBS) is a treatment that applies pulses to the brain across the head of the patient using electrodes. There are many types of NIEBS such as transcranial direct current stimulation (tDCS) which is a form of neuro-stimulation which uses constant, low current delivered directly to the brain area of interest via small electrodes. There are different types of tDCS: anodal, and cathodal. The anodal stimulation is positive (V+) stimulation that increases the neuronal excitability of the area being stimulated. Cathodal (V−) stimulation decreases the neuronal excitability of the area being stimulated. Cathodal stimulation can treat psychological disorders that are caused by the hyper-activity of an area of the brain.

Another form of NIEBS is transcranial alternating current stimulation (tACS) which is a noninvasive means by which alternating currents applied through the skull over the occipital cortex of the brain entrains in a frequency-specific fashion the neural oscillations of the underlying brain. Another class of NIEBS is transcranial pulsed current stimulation (tPCS).

tPCS is a noninvasive method that employs a waveform for use in NIEBS. A tPCS generator is a self-powered device that implements either a fixed tPCS therapy program with preset parameters, or a programmable device that can receive a tPCS therapy program based on treatment options determined by a healthcare professional to be of use to a person with a specific condition. tPCS may also employ a chaotic system that varies many of the pulse characteristics in a random, non-repetitive process.

The present technology is not limited to one form of NIEBS. Therefore, as used herein, NIEBS may refer to many varieties of NIEBS including, but not limited to, transcranial direct current stimulation (tDCS), transcranial alternating current stimulation (tACS), tPCS, and any other neuro-stimulation type protocols such as random noise stimulation and chaotic noise stimulation.

NIEBS involves brain stimulation by low current low voltage that may use alternating square waves or other waves. The effect is to improve the brain's “plasticity,” making it easier to learn. The effect may also be described as an increase in focus, getting into the flow, or being in the zone.

The present technology employs hardware for NIEBS that attaches electrodes to the head of the patient. The hardware may also include speakers such as headphones. The hardware draws power from an audio source such as a digital music player. For example, an mp3 player or a smart phone with a headphone jack may be employed. The hardware of the present technology is able to plug into a standard headphone jack and receive an audio signal to play audio and also is able to draw power for the electrodes for use in NIEBS. The present technology may apply NIEBS to a user and may or may not simultaneously play audio for the user via speakers such as headphones. The pulse for the NIEBS may or may not be based on the rhythm or beat of the audio signal.

The technique of the present technology uses a hardware device with an audio output that may be referred to as a phone connector or audio port. The audio output on the hardware device outputs an audio signal with a measure of alternating current. The audio signal may be for music, speech, or other forms of audio that are to be played on speakers. The present technology is for a device such as a dongle that draws power from the audio port and audio signal of the hardware device. The device or dongle of the present technology may be described as self-powering hardware which uses a rectifier to convert the audio signal into a current such as direct current (DC) power source. The direct current may then be used to generate a NIEBS signal for NIEBS at electrodes associated with a user using a suitable current. The self-powering hardware may also play the audio in earphones. Thus a user of the present technology may employ hardware specific to the present technology with existing or off the shelf hardware such as a smart phone, digital music player, wrist watch with and audio output, iPod, or iPhone without the need to provide an independent power source for the hardware or dongle of the present technology such as batteries. Specifically, the device of the present technology is not powered using sound waves that are generated by speakers, but rather draws power from the audio signal output from an audio port on an existing hardware device.

The present technology may employ NIEBS techniques with electrical current that is safe to use outside the care of a physician and outside a clinical setting. However, the present technology may also use NIEBS techniques that require the oversight of a healthcare profession or physician in a clinical setting. For example, direct current applied directly to a patient in small doses may be unsafe outside of a clinical setting. The present technology may be used in such a clinical setting and may use techniques that combine various types of electrical current for use in NIEBS.

In one embodiment, a digital audio track may be modified to add a Power Tune. The modified digital audio track may be in a standard format such as an mp3 file or other digital file. The Power Tune adds a signal to the audio signal that may control the pulse for NIEBS. For example the modified digital audio track may send a signal that is for the unmodified audio and a signal for a tune that is in or around 20 kHz. A tune in the range of 20 kHz is above the range of human hearing. Thus a user will not hear the 20 kHz tune in the speakers whether it is playing or not. However, the 20 kHz tune may be used to control the pulse of electricity in the electrodes for the NIEBS. The audio track may be modified to include a power tune on the fly during a playback of the audio track or may be done prior to playback of the audio track.

### Embodiments for Systems and Methods for Noninvasive Electrical Brain Stimulation with Power Tunes

Basic Configuration with Power Derived from Audio Source

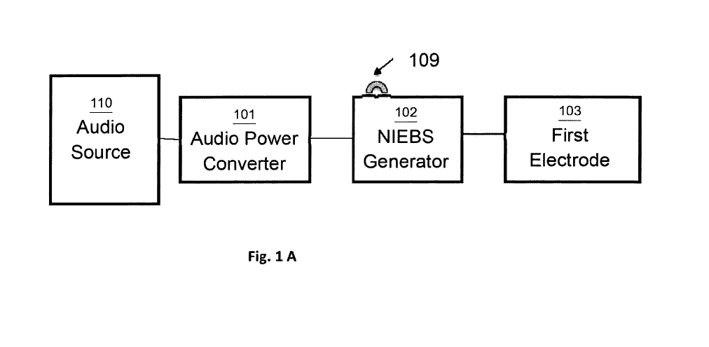

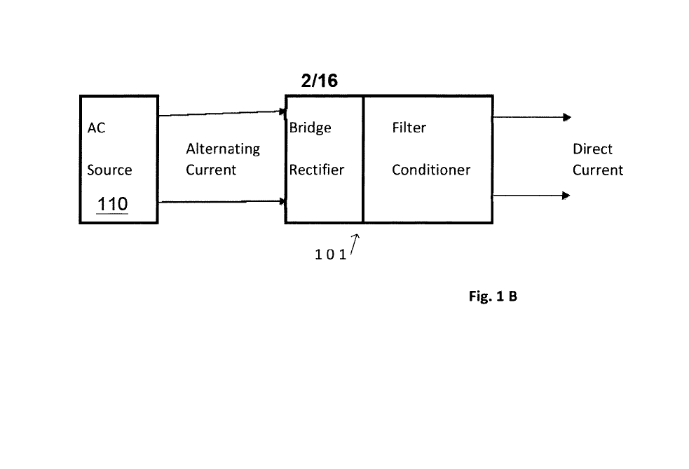

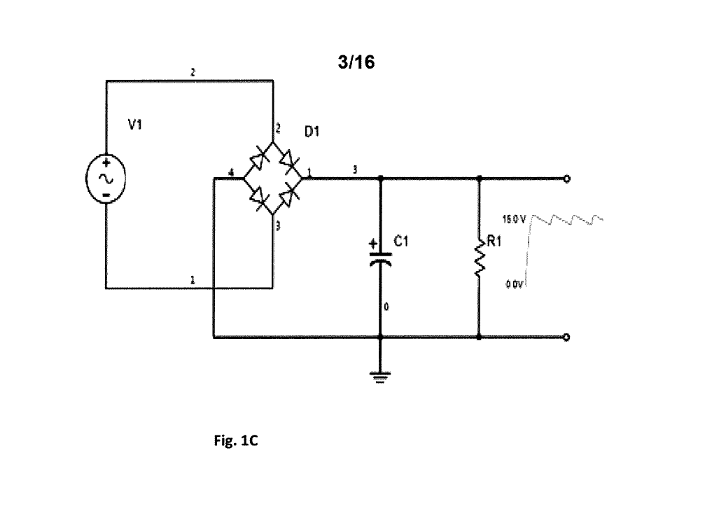

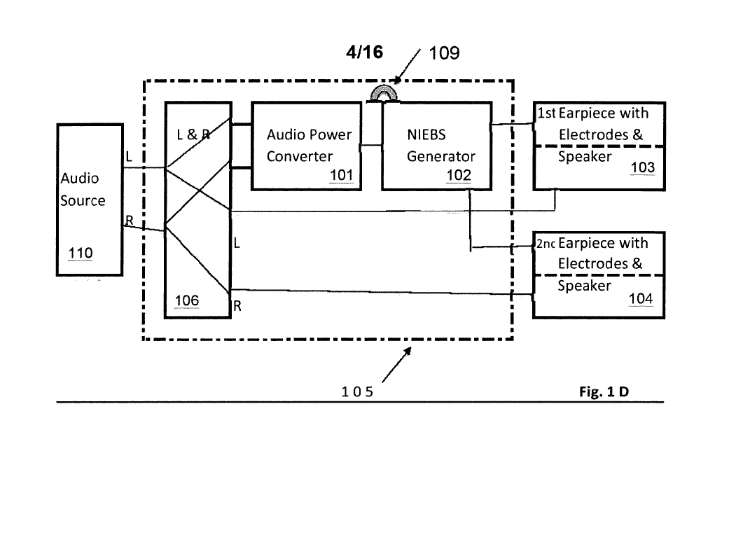

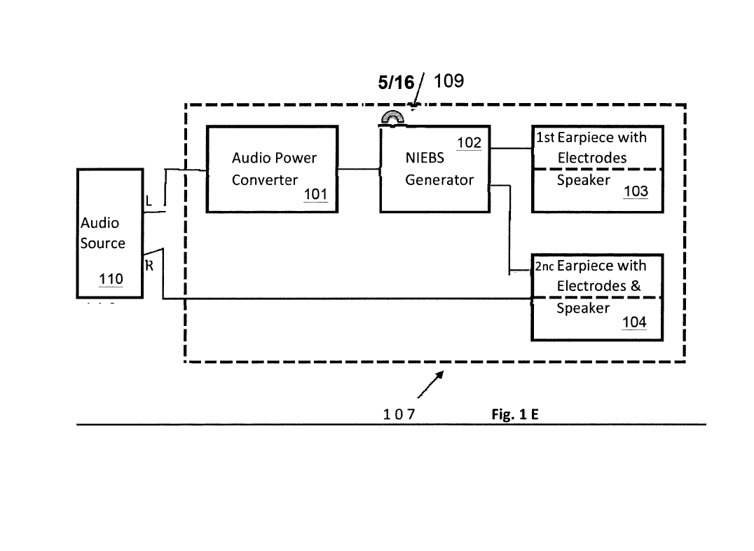

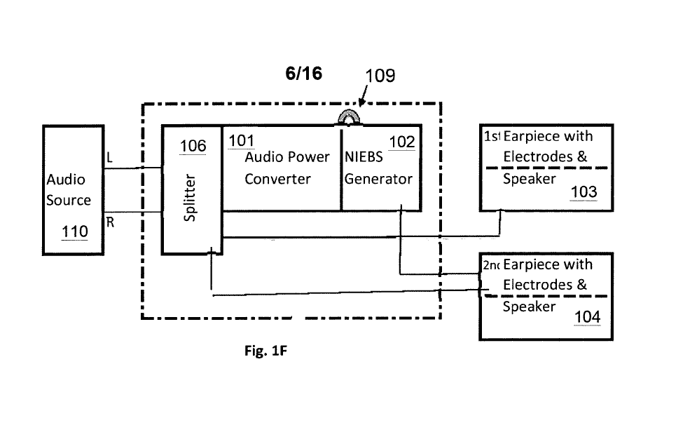

With reference to FIG. 1A. In one embodiment, audio source 110 is a hardware device such as a digital music player, smartphone, or a computer that has an output for an audio signal such as a standard 3.5 mm headphone jack. The audio source may comprise inaudible power tracks. Audio source 110 may be an off the shelf device that is not developed or manufactured specifically for the present technology. The output is able to send electronic signals for audio to play on speakers such as headphones. In FIG. 1A, audio source 110 includes an audio track in a format such as an mp3 format or other digital format. It should be appreciated that audio source 110 is not required to be a digital music player but could be a device that plays audio from an analog source such as a cassette tape player. In one embodiment, audio source 110 is a compact disc player. In one embodiment, the audio source does not have an inaudible portion. The power track has information that will cause the speakers connected to 110 to play audio at a high frequency such as 20 kHz that is inaudible to the human ear. However, the modified audio track may not be sent to the speakers. For example, the power track may comprise one channel of audio that is not sent to the speakers or may be split after the signal for the modified audio track leaves audio source 110 such that the power track is only sent to audio power converter 101 and NIEBS generator 102 and is not sent to the speakers for playback. This is more clearly demonstrated by FIG. 1E.

In one embodiment, audio power converter 101, NIEBS generator 102, and intensity adjust 109, comprise a single hardware unit for the present technology. The single hardware unit may be described as a dongle. The dongle may have a wire to connect with audio source 110, for example the wire may include a standard connector for a 3.5 mm headphone jack. In one embodiment, the dongle has a port or a plurality of physical ports for outputs. For example, 1^{st} earpiece with electrodes 103 may be able to connect with NIEBS generator 102 via a wired connected using standard or proprietary connectors. There may be more than one physical output port such that music or audio may be sent over one or more ports and powered signals for the NIEBS are sent over another channel. First electrode 103 may be hardwired to the dongle or NIEBS generator 102. As described above, the hardware device or unit 110 may be an off the shelf preexisting device that was not created specifically for the present technology. In one embodiment, audio source 101 is able to draw or receive power from the audio output of audio source/AC source 110 and convert the power to a usable form for NIEBS generator 102. For example, audio source 101 may convert alternating current AC to direct current DC.

NIEBS generator 102 is then able to generate a signal for NIEBS to be applied to a user via first electrode 103. In one embodiment, the signal generated by NIEBS generator 102 is based on the audio track from audio source 110 such that the beat and pulse of the audio track control the pulses of the NIEBS signals. The intensity or amplitude of the NIEBS applied to the user via first electrode 103 is controlled via intensity adjust 109. For example, intensity adjust 109 may be a physical wheel that may be adjusted by a user. Intensity adjust 109 may also be buttons or other types of controls. There may be other controls to control other aspects of the signals such as pulse duration, pulse polarity, period between pulse trains, etc. A pulse train is defined to be a series of waves or pulses for the NIEBS signal.

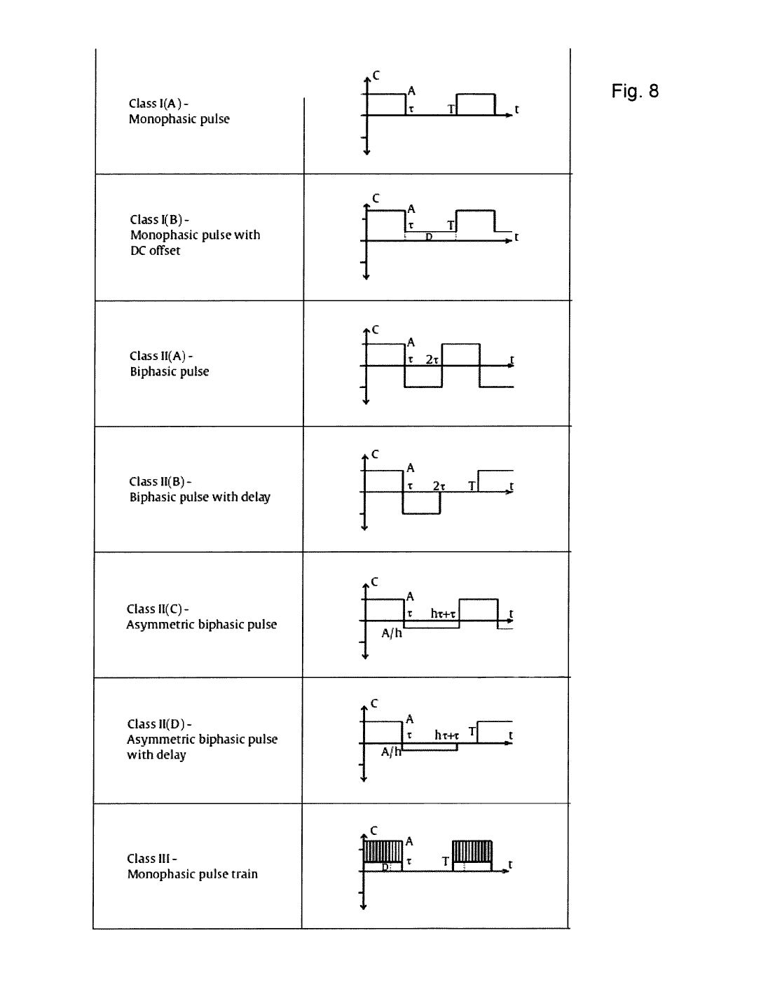

NIEBS generator 102 generates signals in the form of electrical pulses that have a wave shape. For example, the wave shapes may be similar to those depicted in FIG. 8. NIEBS generator 102 may comprise or be connected to a library of sorts that defines waves or pulse trains that are to be generated for use in the NIEBS therapy. For example, NIEBS generator 102 may have a memory or storage module associated with it. Such a memory may be updated or changed.

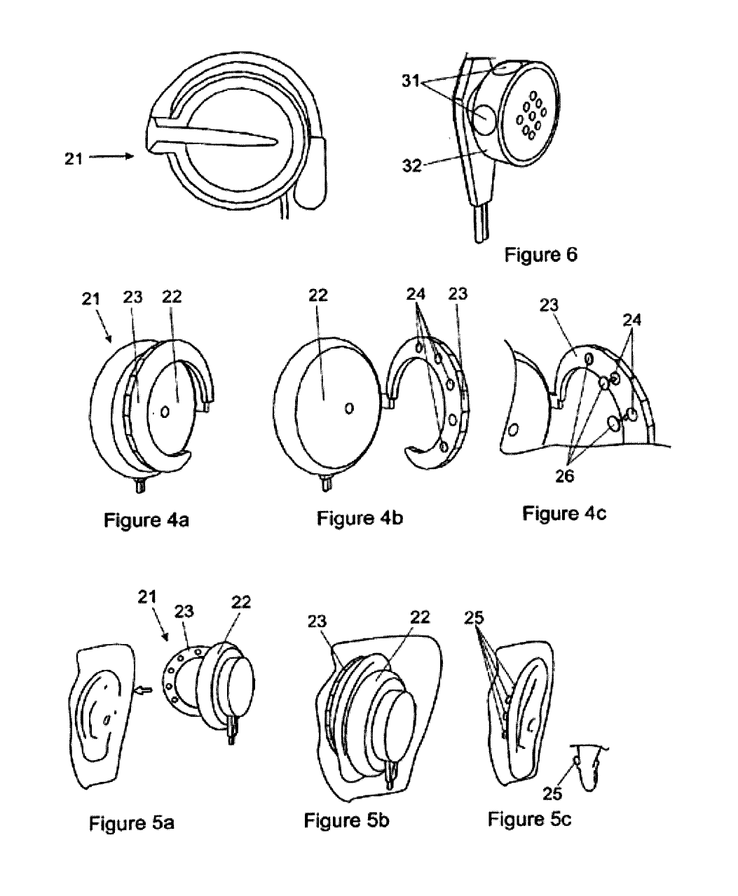

In one embodiment, 1^{st} Earpiece with electrodes 103 comprises electrodes that attach to a user's heard. For example, the electrodes may clip onto the users ear or otherwise be applied to the skin. 1^{st} earpiece with electrodes 103 may comprise any number of electrodes. 1^{st} earpiece with electrodes 103 does not require the electrodes to attach to the ear. 1^{st} earpiece with electrodes 103 may be hardwired to NIEBS 102 or may be separate and attached via ports. The speakers which may or may not be associated with 1^{st} earpiece with electrodes 103 may or may not be coupled into one device or frame with the electrodes. In other words, the speakers may be separate from the electrodes. Speakers that are separate from the electrodes may be wired or connected directly to audio source 110 or may be wired or connected to NIEBS generator 102. 1^{st} earpiece with electrodes 103 may be one of the embodiments depicted in FIGS. 4A-6.

The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

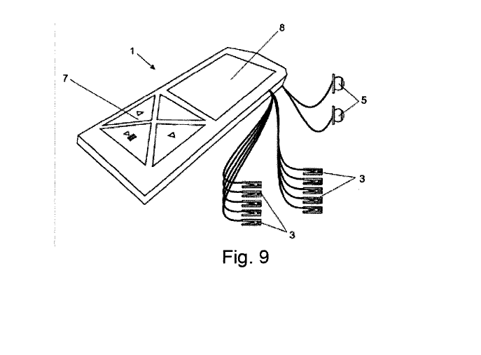

103 may include only electrodes or may include both electrodes and speakers. If 103 includes both electrodes and headphones, the electrodes may be combined with the housing or frame of the headphones as is depicted in FIGS. 4A-C, 5A-C, and 6 or the speakers and electrodes may be separate as is depicted in FIG. 9. FIG. 9 depicts device 1 which may be a dongle that connects with the standalone off the shelf hardware device that outputs audio. Device 1 may or may not include display 8 and buttons or controls 7. The buttons or controls 7 may be for controlling the intensity or other parameters of the NIEBS signal. Headphones 5 may or may not be included and may or may not be hardwired to device 1. The headphones 5 may be detachable and replaced with off the shelf headphones. The electrodes 3 depict 10 different electrodes. However, any number of electrodes may be employed. In a typical embodiment, at least two electrodes are required to complete an electrical circuit.

Referring now to the nature of the sets of multi electrodes, each set is made, in this embodiment of the invention, in the form of a multi electrode unit that is illustrated in FIGS. 4A to 5C of the accompanying drawings. In this instance an earphone unit (21) has an earphone (22) of the type shaped to be held against the ear a short distance outwards from the entrance to the auditory canal and an arcuate electrode carrier (23) that can be swung outwards away from the sound emitting face of the earphone.

The electrode carrier can thus be swung inwards to engage the rear face of the pinna of a person's ear to hold the earphone unit in position. In the operative position the multiple electrodes (3) in the face of the arcuate electrode carrier contact the rear of the pinna of the ear at arcuately spaced positions that are indicated by numeral (25) in FIG. 5c. Each individual electrode is preferably covered by an electrically conductive felt patch (26) or the like, as shown in FIG. 4c.

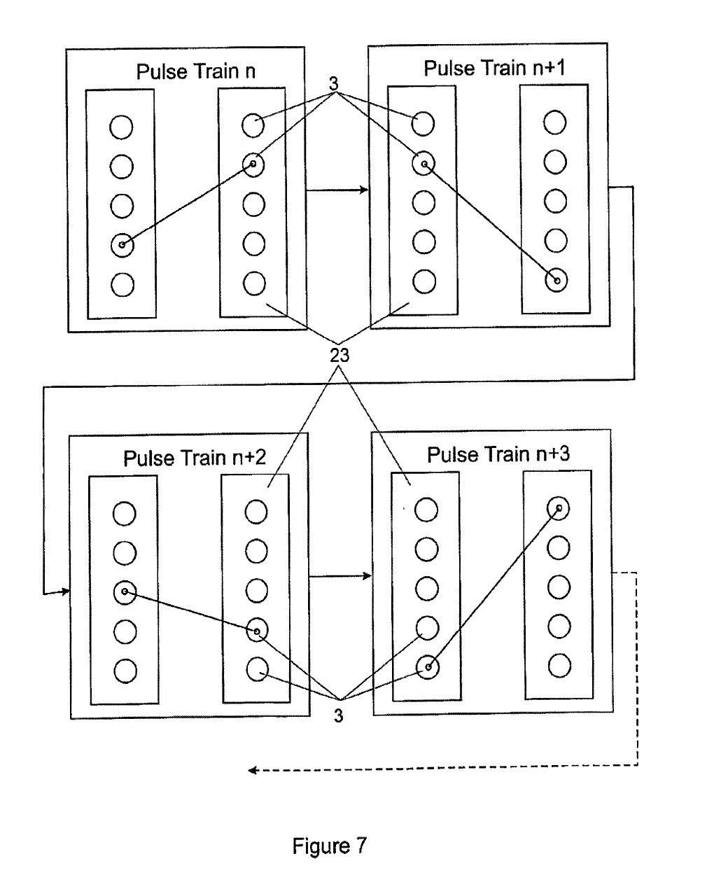

In this variation of the invention, the arrangement is such that current is only induced between one electrode of each earphone unit at any one time and different pairs of cooperating electrodes are selected sequentially or randomly via the microprocessor. FIG. 7 indicates some sequential connections between single electrodes of each multiplicity thereof simply by way of example. The microprocessor is a component o for associated with NIEBS generator 102.

With reference to FIG. 1B, there is illustrated an audio power converter with options of music and other audio. In one embodiment, AC source 110 provides an alternating current source that may be low powered. For example 110 may be a digital music player or smartphone. The alternating current output by 110 may be a current designed to play music or other audio at speakers such as headphones. Audio power converter 101 is able to receive the AC current and convert it to direct current DC using a bridge rectifier and a filter conditioner. 101 may be a hardware device well known in the art.

With reference to FIG. 1C which depicts a schematic diagram for power converter and filter 101 which may have the same features and capabilities of audio power converter 101 of FIG. 1B.

With reference to FIG. 1D there is illustrated a dongle 105 as an assembly holder for items. Audio source 110 may be the same as 110 in FIGS. 1A and 1B. Audio source 110 is capable of generating an audio signal output. Audio source 110 may be an off the shelf preexisting device such as a smart phone. In FIG. 1D audio source generates an output signal that comprises two channels, a left and a right depicted by L and R. L and R may be split by splitter 106 which sends a portion of channel L to speaker 103 and a portion of channel R to speaker 104. Splitter 106 also sends a portion of R and a portion of L to audio power converter 101 which converts power from L and R to a form suitable for NIEBS. NIEBS generator 102 then generates a NIEBS signal based on L and R that is for NIEBS. The intensity of the NIEBS signal may be controlled by a user via intensity adjust 109. NIEBS generator 102 then sends the signal to earpieces 103 and 104. Earpieces 103 and 104 are able to apply NIEBS to the user via electrodes that apply electric pulses to the skin of the user while simultaneously playing the audio from audio source 110 via the speakers. The audio may be any type of audio such as music or speech. The NIEBS signal may be based on the rhythm and beat of the music associated with the audio or may be based preprogrammed data stored in memory associated with NIEBS generator 102.

Splitter 106 may split the power tune associated with a modified audio track away from the audio track such that the power tune is sent to audio power converter 101 and the original audio track is sent to 104.

103 and 104 of FIG. 1D each comprise both an electrode and a speaker. In one embodiment, 103 and 104 combine the speaker and the electrode into one component that clips to an ear. In one embodiment, the speaker and the electrode are separate.

Dongle 105 refers to the following components: splitter 106, audio power converter 101, NIEBS generator 102, and intensity adjust 109. These hardware components may all be encapsulated or housed together and referred to as a dongle. The dongle may have a cable or wire that connects to the output of audio source 110 and cable or wires that connect to earpieces 103 and 104. Earpieces 103 and 104 may be hardwired to dongle 105 or dongle 105 may have ports for the speakers and electrodes. There may be a single port or a plurality of ports.

With reference to FIG. 1E. Audio source 110 may be similar to 110 of FIGS. 1A-1E. In one embodiment, audio source 110 outputs two channels L and R. Available audio passes to 2^{nd} earpiece. Optional frame assembly holder for all items 107. FIG. 1E depicts channel R being sent directly to 104 whereas channel L is first received by the 2^{nd} earpiece with electrodes 101 where a portion of the power is converted for use in NIEBS. NIEBS generator 102 then sends a NIEBS signal to both earpieces 103 and 104. Audio power converter 101 and NIEBS generator 102 may also allow a portion of the signal from channel L to pass to the speaker of earpiece 103 to play the audio of channel L. Thus, in the embodiment of FIG. 1E the speakers of earpieces 103 and 104 may play audio in stereo whereas the electrodes of earpieces 103 and 104 apply NIEBS based only on the audio from the L channel. In one embodiment, audio power converter 101 and NIEBS generator 102 may use the R channel instead of the L channel.

In one embodiment, the original audio track is sent to the speakers via the R channel and the power track may be sent to 101 via the L channel. The R channel may be used to play audio in two speakers and may be mono. The modified track may be created such that one channel of the modified track has the power tune and one channel has audio that is in the audible range such as music or speech.

In one embodiment, audio source device 110 is a device that has a software program such as an app that can modify any audio track to include a power tune. The modified audio track may or may not be created and stored on device 110. Such an app may be downloaded from a third party is made to be compatible with an off the shelf device 110. The software program may modify any audio track on the fly during playback of the audio device. The software program has a library of power tunes that may be updated via the Internet or other data connections. A user may be able to use the software applications to control which power tune is used to modify any audio track.

With reference to FIG. 1F, there is an illustrated integrated power/CES generator which depicts audio source 110 generating an audio output and splitter 106 splitting the signal such that a portion is delivered unchanged to earpieces 103 and 104 and another portion is delivered to audio power converter 101 to convert the power of the signal. NIEBS generator 102 then generates a NIEBS signal based on the audio outputs and sends the NIEBS signal to the electrodes of earpieces 103 and 104. FIG. 1F depicts an embodiment where splitter 106, audio power converter 101, and NIEBS generator 102 are all components of the same hardware chip. Such as chip may be housed in a dongle as indicated by the dotted line. Moreover, such a chip may be a produced and marketed to third parties for use in building their own devices in accordance with the present technology. For example, such a chip could be placed in a device such as a digital music player to add NIEBS functionality to the digital music player. In such an example the chip is hardwired into the device.

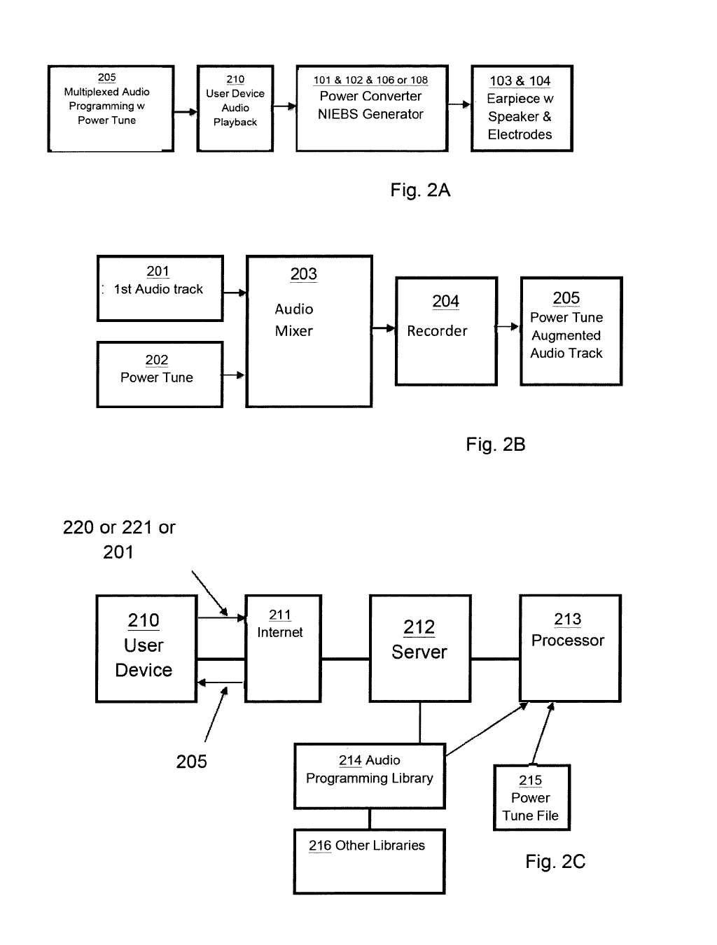

With reference to FIG. 2A. 101, 102, 103, 104, 106, and 108 are similar to what is depicted and described for FIGS. 1A-F. Element 205 depicts a multiplexed audio programming with a power tune. The power tune comprises data that when played via device 210 and speakers 103 and 104, the audio will be inaudible to human ears. For example, the power tune may be at or around 20 kHz. Element 205 also comprises audio that is audible to humans. For example, element 205 may be an audio track that is in mp3 format that plays an audio track audible to human ears but has been digitally combined with a computer system to also play the power tune simultaneously. 210 is a user device that is similar to 110 as depicted and described for FIGS. 1A-F. The power tune is designed to be used with the present technology for powering a NIEBS generator. For example, the power tune may be used by a converter to convert the AC signal from the audio source to a DC current for use in the generation of a NIEBS signal to be applied to a user via electrodes. The NIEBS generator then controls the pulses of the NIEBS signal such as the duration, intensity, frequency, etc.

Elements 205 and 210 may represent an app executing on device 210 that is able to create a modified audio track that includes a power tune using any audio track associated with audio source device 110. Such an app may create and store the modified audio track on audio source device 110 or may create the modified audio track on the fly each time the unmodified audio track is played.

With reference to FIG. 2B which depicts the creation of 205 which is a power tune augmented audio track. 201 represents an audio track in a digital format that is unmodified. 202 represents a power tune that is inaudible to the human ear. Audio mixer 203 is able to mix audio track 201 and power tune 202 together. For example, audio mixer 203 may be a computer system or may be an actual audio player with speakers that audibly plays both of audio track 201 and power tune 202. Recorder 204 then records the output of audio mixer 203 to combines audio track 201 and power tune 202 to form power tune augmented audio track 205. Recorder 204 may be a digital recorder and may be associated with a computer system. Recorder 204 may comprise a microphone to record the audible sounds of audio mixer 203 or may digitally combine the digital output of audio mixer 203. Audio mixer 203 may be an app associated with device 110.

With reference to FIG. 2C. A user of the present technology may desire to use a power tune or combine a power tune with an audio track that favored by the user. However the user may not have access to power tracks or may lack to ability to combine power tunes with unmodified audio tracks. FIG. 2C depicts an environment that may assist a user in combining a favored audio track with a power tune. User device 210 may send an unmodified audio track via Internet 211 to a server 212 associated with processor 213. Alternatively, user device 210 may request an audio track be combined with a power tune where the user does not supply the audio track. If the user does not supply the audio 213 then accesses audio programming library 214 or other libraries 216. For example, audio programming library 214 or other libraries 216 may be a privately owned library or may be a 3^{rd} party library such as iTunes or other commercial service for purchasing audio tracks. Processor 213 then combines the audio track with the power tune file 215 to create power tune augmented audio track 205 which is returned or downloaded to user device 210. Processor 213 implements 203 and 204. Power tune file 215 may be supplied by the user or may be supplied by a third party. Server 212 and processor 213 may be a 3^{rd} party separate from the user associated with user device 210. Such a 3^{rd} party may be a service to provide a user with combined tracks 205.

### Hardwired Factory Set

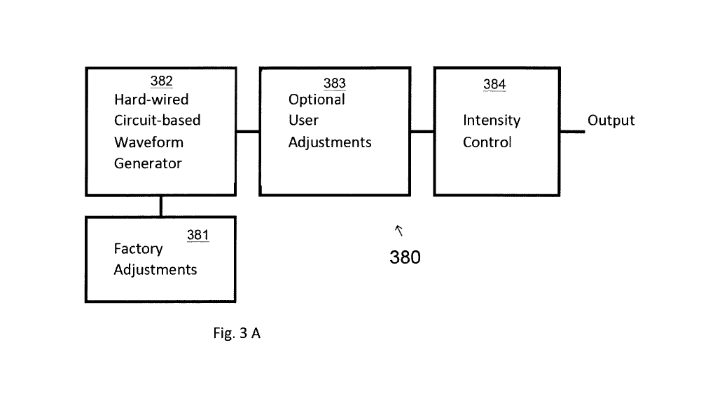

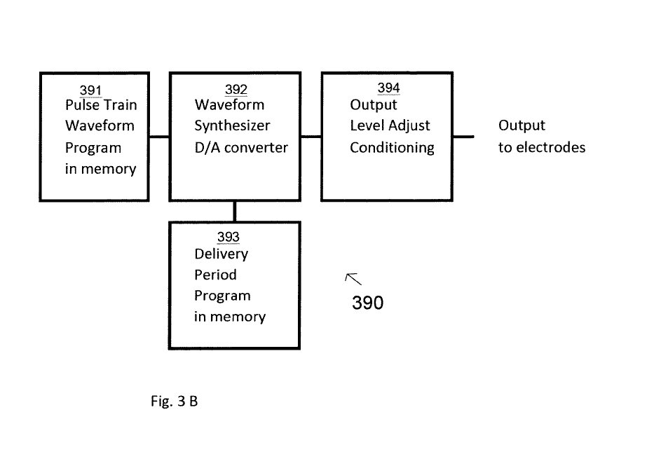

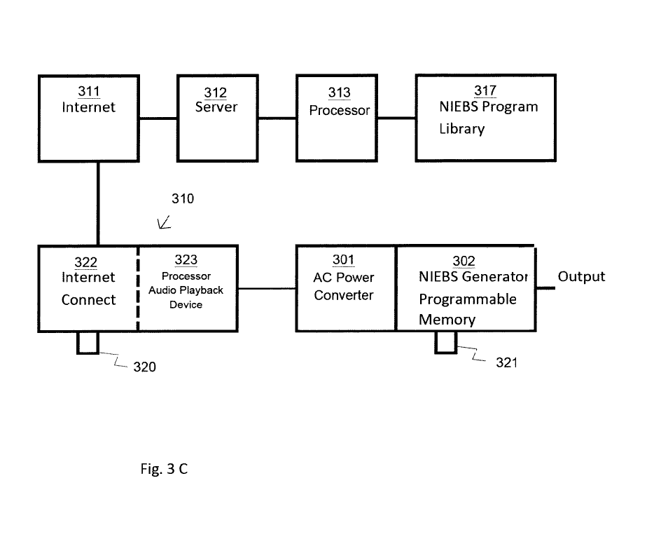

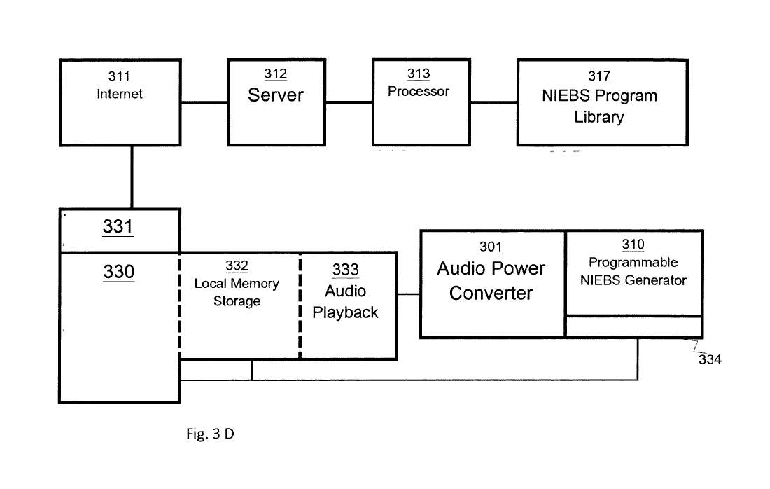

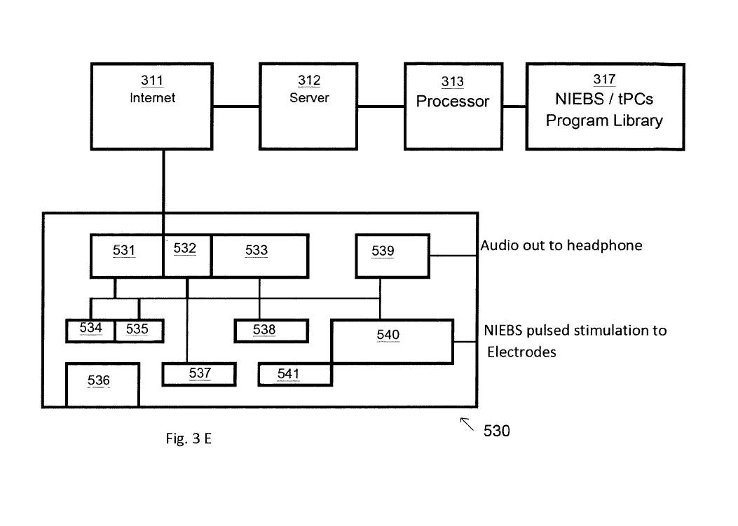

With reference to FIG. 3A hardwired circuit based waveform generator that can be adjusted with optional user adjustments and intensity control. FIG. 3A may refer to a prior art solution for NIEBS. The hardwired circuit 380 may be adjustable at the factory during a manufacture process. The solution of FIG. 3A may include hard-wired circuit-based waveform generator 382, factory adjustments 381, optional user adjustments 383, intensity control 384, and an output.

With reference to FIG. 3B there is provided a programmable NIEBS system, which depicts an embodiment of the present technology with a waveform synthesizer D/A converter 392. This allows the synthesizer to access a pulse train waveform program in memory and then synthesize a waveform. The delivery period program may be in a memory associated with the synthesizer. The synthesizer may download or update the pulse train waveform program or the delivery period program. The output level may adjust the conditioning. Essentially the synthesizer is able to read a waveform file and run it into a digital-to-analog converter (D/A). The present technology may operate to update the waveform file and the delivery period file as appropriate and tag them all with names and ID numbers. The components or modules depicted in FIG. 3B are for a programmable NIEBS generator 390. Such a generator may have the same capabilities and features as those described for 102 of FIGS. 1A-F. FIG. 8 depicts examples of waveforms that may be employed by programmable NIEBS generator 390.

With reference to FIG. 3C which shows how an update process in the general case works to update programmable NIEBS generator 390 of FIG. 3B. The process may include a NIEBS data download. Such a process may work with any kind of computer [laptop, desktop, handheld pda/cellphone] where there is an audio output for outputting audio to drive the Power converter 301 and thus power the NIEBS generator 390. FIG. 3C depicts the embodiment of an independent NIEBS device, perhaps integrated with a headset. 310 depicts the device for generating an audio output. AC power converter 301 and NIEBS generator programmable memory 302 depict the hardware of the present technology that is power only by the audio source and output NIEBS signals to a headset and may or may not output audio as well. 302 has programmable memory that may be updated or changed via data port 321 that may be a standard port that connects with a wired connector. Data/power port 321 may also be a wireless device using techniques such as near field communications, Bluetooth or Wi-Fi. Data/power port 321 may connect with a local device such as a personal desktop or laptop computer or a smartphone. The local device is then able to use internet 311 to contact server 312 and processor 313 to obtain programs for NIEBS signal formulations and then send such NIEBS signal formulations to NIEBS generator programmable memory 302 via data/power port 321. NIEBS program library is a database comprising a variety of programs and data for NIEBS generator programmable memory 302. Thus the program data in memory 302 may be updated, replaced or added to. Such updating or other exchanges of data may occur automatically on a periodic basis or may be pushed via server 312. The update may be described as updating firmware for the NIEBS generator. Elements 301 and 302 may be an integrated and stand alone unit. The update process of FIG. 3C may also use NIEBS Program Library 317, internet connect element 322 having a data/power port 320, and processor audio playback device 323. NIEBS generator programmable memory may have an output, a data/power port 321, and an intensity control (not shown). The connection to the internet may be made via any computer. Any audio player may be used.

### Automated Update of NIEBS Signal Formulations Via an Internet Connection

With reference to FIG. 3D which shows how a cellphone-based system with a separate NIEBS power/generator would look in particular. FIG. 3D relates to a program update system for PDA & stand alone NIEBS and a stand alone NIEBS/power converter operating with a modern cellphone. In other words, FIG. 3D depicts a cell phone device or other handheld device that is capable of cellular communications and uses those cellular communications to update the programs for programmable NIEBS generator 310 which is part of stand-alone dongle 308. The method of updating or exchanging data is similar to that which is described in FIG. 3C.

With reference to FIG. 3E which shows what an integrated system might look like; using the cellphone model, install the NIEBS generator in it, and use power from it for the NIEBS generator. FIG. 3E relates to an integrated PDA/NIEBS system with updating. In other words, FIG. 3E depicts a cell phone device or other handheld device that is capable of cellular communications and has components associated with the present technology built in or hardwired in to one device. For example, device 530 has a phone module for communications as well as Wi-Fi and other components associated with a smart phone or cell phone. However 530 also comprises program NIEBS generator. The update may be described as updating firmware for the NIEBS generator. The system may include internet 311, server 312, processor 313, and NIEBS/tPCs program library.

A cellphone 530 may be a modern cellphone and may contain the following elements: phone 531, Wi-Fi 532, processor 533, ROM 534, RAM 535, audio tracks 536, battery 537, display 538, audio speaker 539, programmable NIEBS generator 540, and controls 541.

With reference to FIG. 8 which depicts wave forms that may be employed for use with the present technology. A NIEBS generator may receive wave forms from an audio source or from a waveform synthesizer associated with the NIEBS generator. The NIEBS generator may generate a NIEBS signal with associated wave forms for the NIEBS treatment. FIG. 8 depicts well known square wave forms for use in the present technology. The present technology is not limited to wave forms in FIG. 8 but may also employ other wave forms such as sine waves.

Wave forms for the present technology may be stored in a library and are used to create pulse patterns or pulse trains for use in NIEBS. The wave forms may be implemented via a programmable D/A converter. Research indicates that different pulse patterns have different effects on the brain, and that some pulse patterns have different effects on various conditions. Therefore, there is a need for a library of different pulse patterns to suit different health conditions.

The rate of pulses per second refers to a start of positive-going pulse to stop, with the delay until the next positive-going pulse starts. Like a sine wave, regardless of whether or not there is a negative-going pulse. “Beginning of a pulse rising, to the next time the pulse starts rising again.” The following are examples of pulse rates that may be employed by the present technology:

1. Pulse rate in range of 3-5 Hz. Low Freq.

2. Pulse rate in range of 50-100 Hz. Low Freq.

3. Pulse rate in range from 100-640 Hz. High Freq.

4. Pulse rate in range of 0.1-100 Hz

5. Direct current

Current level delivered: 1.5 mA. [milli-Ampere]

Current density on the skin: safety limit is between 25 and 60 microA/cm² [from Poreisz et al., 2007] The electric field across the brain tissue is on the order of less than 5 mV/mm, or 5 milli-Volts/millimeter.

Pulse pattern may be a Random Noise Stimulation pattern. Good results reported by Fertonani et al in paper “Random Noise Stimulation Improves Neuroplasticity in Perceptual Learning,” The Journal of Neuroscience, Oct. 26, 2011 31(43):15416-15423.

Noninvasive electrical brain stimulation (herein referred to as NIEBS) applies gentle micro-current pulses to the brain using electrodes. The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

In NIEBS significant amounts of current pass the skull and reach cortical and subcortical structures. In addition, depending on the montage, induced currents at subcortical areas, such as midbrain, pons, thalamus and hypothalamus are of similar magnitude than that of cortical areas. Incremental variations of electrode position on the head surface also influence which cortical regions are modulated. The high-resolution modeling predictions suggest that details of electrode montage influence current flow through superficial and deep structures. Also, laptop based methods for tPCS dose design using dominant frequency and spherical models. These modeling predictions and tools are the first step to advance rational and optimized use of tPCS and NIEBS.

It is widely accepted that NIEBS stimulates the brain to manufacture neurotransmitters, like endorphins, which improve moods, emotions and cognitive capabilities. Noninvasive electrical brain stimulation has also been proposed for treatment following a stroke, brain trauma, high blood pressure, and Alzheimer's disease, as well as any or all neurological disorders, any or all mental disorders, and any or all cognitive enhancements. The present technology may also be used by healthy users or users who are not suffering from any diagnosed disorders or diseases. For example, a healthy user may be a student using the present technology to increase focus and learning abilities or may be an athlete using the present technology to increase sports performance.

The signals apparently normalize the electrical output of the brain. NIEBS has thus been used or tested to treat substance dependence, depression and anxiety. It has been noted in at least some instances that NIEBS has equal or greater efficacy for the treatment of depression when compared to antidepressant medications, with fewer side effects. NIEBS may be used specifically in combination with anti-depressant drugs and may be used to eliminate the side effects of central nervous system (CNS) medications or drugs in general. NIEBS may also be used in conjunction with other traditional medicine.

Treatments can be used in association with the present technology in ranges from less than one second up to an infinite number of seconds. The present technology is not limited to a particular range of duration, current, or frequency. The following ranges are meant as examples and do not limit the present technology. In one embodiment, a range is used from 10 to 30 minutes in duration although the treatments may extend up to 11/2 hours depending on the electrical current configuration. The currents employed may be applied in pulse form or direct form with a pulse width in the range of from about 1 to about 500 milliseconds (ms) at a frequency of from about 0.1 Hertz (Hz) up to 1000 Hz with the current being less than 1 milliampere (mA) up to 5 mA

In accordance with an embodiment of the invention there is provided equipment for the implementation of a method as defined above, said equipment comprising a noninvasive electrical brain stimulation pulse generator and associated electrodes for applying pulses generated by the pulse generator to the head of a patient, WHEREIN the equipment includes multiple electrodes.

In an embodiment of the invention, there is an audio signal player and at least one associated loudspeaker for converting output from the signal player into audible sound. The at least one loudspeaker is preferably a pair of earphones and the noninvasive electrical brain stimulation pulse generator and sound signal generator may be built into a single unit, but are not necessarily thus combined.

Note that there are the following types of stimulation configurations:

1. Positive going pulse, with a direct current average in one direction. Class 1A and Class 1B deliver a varying amount of direct current in little bursts.

2. Alternating current pulses, where the direction of current alternates from positive going to negative going, as in Class IIA and Class IIB and IIC and IID. The average may be in one direction predominantly, or may average out to zero if the pulses are symmetric and equal in duration over time. You can see that for some modes, there is a net direct current passing thru the brain.

3. Class III shows a pulse train with a delay between delivery of a series of pulses.

The next paragraphs discuss how this delay may be configured, and is part of the overall therapy formulation that is available to a medical practitioner.

1. Random time period. Use a random number generator with a specified range in seconds. For example, 1-100 seconds. Run the random number generator which is set to produce a number between 1 and 100. Use that number as the time period between pulses. Run the generator after each pulse to determine the next time delay, or period, from the last pulse.

2. Semi-random time period.

Pick some time periods that are known to have some therapeutic effect. Make a table. For example:

Random No. 1 3 5 10 20 40 60 100.

Bin containing 1 2 3 4 5 6 7 8

the delay

It can be anything, this is just an example.

Then randomly select from this group of time periods. Again, use a random number generator whose bounds are the number of allowed states. In the above example, there are 8 possible delay time periods. Set the random number generator to select any of the numbers from 1 to 8. Use the time delay associated with that bin number.

Say the random number generator picks 4. That means we use 10 second delay as the time period to the next pulse train initiation.

3. Periodic but increasing delay, with a plan

Here the time delay from one pulse train event to the next is arbitrarily set to predetermined sequence. It may be one with a set increase from one period duration to the next. As in 5 10 30 60 repeat 5 10 30 60.

4. Periodic, static period

Set delay to one of the group [1, 2, 3, 4, 5, 6, 7, 8, 9, 10] seconds. Or any other time period from 1 to 300 seconds, for example.

5. Continuous pulse train with no delay between any arbitrary group of pulses. Arbitrary duration of such pulse trains, selected from group [1-1000] seconds.

6. Direct Current Stimulation

No pulses, just application of a constant voltage for some time period. One could consider this a special case of a single positive going pulse with a really long time duration.

### Notes on Using Chaotic/Random Pulse for NIEBS:

Pulses or pulse trains for NIEBS may be patterned or random. However, the idea of random pulses may not be desirable as random may still indicate a measurable structure impulse. The term chaotic pattern is better description of the pulse referred to herein. Chaotic may also be used to define the variety of the pauses or periods in between pulse trains. The level of chaoticness may be controlled via a controller similar to 109 of FIG. 1A.

An example noninvasive electrical brain stimulation system is illustrated in FIG. 9 which includes a device 1, comprising a display 8, controls 7, headphones 5, and electrodes 3.

### Computer Implemented Methods

It should be appreciated that the methods described herein may be computer implemented methods that are carried out by processors and electrical components under the control of computer usable and computer executable instructions. The computer usable and computer executable instructions reside, for example, in data storage features such as computer usable volatile and non-volatile memory. However, the computer usable and computer executable instructions may reside in any type of computer usable storage medium. In one embodiment, the methods may reside in a computer usable storage medium having instructions embodied therein that when executed cause a computer system to perform the method. In one embodiment, the NIEBS signals described herein are non-transitory but rather are sent over wired connections to the electrodes.

It is intended that the foregoing description be regarded as illustrative rather than limiting, and that it be understood that the detailed description should not be used to limit the scope of the invention.

1. A method for providing noninvasive electrical brain stimulation “NIEBS”,
comprising:
receiving a signal at a power converter wherein the signal is an audio signal received from a device;
converting power from said audio signal to a form suitable for NIEBS;
generating a NIEBS signal using said power converted from said audio signal; and
applying NIEBS based on said NIEBS signal to a user via electrodes.

2. (canceled)

3. (canceled)

4. The method of claim 1 wherein an intensity of said NIEBS signal is controlled via a hardware control.

5. (canceled)

6. The method of claim 1 wherein said converting power and said generating said NIEBS signal and said applying said NIEBS is accomplished using only the power received from the device with no other independent power source.

7. A device for applying noninvasive electrical brain stimulation “NIEBS”:
a power converter for receiving an audio signal output from a device and converting a portion of the power from the audio signal output to a form suitable for NIEBS;
a NIEBS generator for generating a NIEBS signal; and
at least two electrodes for attaching to the skin of a user for applying NIEBS to said user based on said NIEBS signal using only power derived from said audio signal output.

8. A method for providing noninvasive electrical brain stimulation “NIEBS”, comprising:
receiving an audio signal wherein the audio signal comprises an audible portion and an inaudible portion;
sending said audible portion is sent to at least one speaker to be played and said inaudible portion to a power converter;
converting power from said inaudible portion to a power form suitable for NIEBS;
generating a NIEBS signal based on said converted power; and
applying NIEBS to a user via said NIEBS signal wherein said NIEBS is powered only by power derived from said audio signal.

9. The method of claim 8, wherein the audio signal is a multiplexed audio program, the method further comprising splitting said multiplexed audio program in order to send said audible portion to at least one speaker and in order to send said inaudible portion to said power converter.

10. The method of claim 8, wherein the audio signal comprises a left channel and a right channel and wherein the inaudible portion is present in only one channel.

11. The method of claim 8, wherein the inaudible portion comprises a signal with a frequency above 20,000 Hz.

12.-15. (canceled)

16. A computer-readable medium comprising computer-readable instructions to implement the method of claim 8.

17. An apparatus for providing noninvasive electrical brain stimulation “NIEBS” using a received audio signal comprising an audible portion and an inaudible portion, comprising:
at least one speaker for playing said audible portion of said received audio signal;
a power converter for converting power from said inaudible portion of said received audio signal to a power form suitable for NIEBS;
a NIEBS generator for generating a NIEBS signal based on said converted power;
electrodes for applying NIEBS to a user via said NIEBS signal wherein said NIEBS signal is powered only by power derived from said audio signal.

18. The apparatus according to claim 17, wherein the audio signal is a multiplexed audio program, the apparatus further comprising a splitter for receiving the multiplexed audio program at the splitter.

19. The apparatus of claim 17, wherein the audible portion and the inaudible portion are on the same audio track.

20.-24. (canceled)

25. The apparatus of claim 18, wherein the splitter further comprises a low-pass filter for filtering out the inaudible portion.

26. A method for creating a multiplexed audio program:
receiving a request for a multiplexed audio program based on a modified audio track;
obtaining an unmodified audio track at a mixer;
mixing said unmodified audio track with a power tune to produce a multiplexed audio program.

27. The method of claim 26, further comprising recording said multiplexed audio program.

28.-31. (canceled)

32. The method of claim 26, wherein the power tune is inaudible.

33. A computer-readable medium comprising computer-readable instructions to implement the method of claim 26.

34. A method for providing NIEBS, comprising:
receiving a multiplexed audio program at a splitter from a device wherein the multiplexed audio program comprises an audible portion and an inaudible portion;
splitting said multiplexed audio program at said splitter such that said audible portion is sent to speakers to be played and said inaudible portion is sent to a power converter;
converting power from said inaudible portion to a power form suitable for NIEBS;
generating a NIEBS signal based on said inaudible portion; and
applying NIEBS to a user via said NIEBS signal wherein said NIEBS is powered only by power derived from said multiplexed audio program.

35. A apparatus for providing NIEBS, comprising:
a splitter for receiving a multiplexed audio program at a splitter from a device wherein the multiplexed audio program comprises an audible portion and an inaudible portion;
at least one speaker for playing said audible portion;
a power converter for converting power from said inaudible portion to a power form suitable for NIEBS;
a NIEBS generator for generating a NIEBS signal based on said inaudible portion;
electrodes for applying NIEBS to a user via said NIEBS signal wherein said NIEBS is powered only by power derived from said multiplexed audio program.

36.-38. (canceled)

39. The apparatus of claim 35 wherein the splitter further comprises a low-pass filter in the second channel for filtering out the inaudible portion.

40.-66. (canceled)
